(19) United States
(12) Patent Application Publication
(10) Pub. No.: US 2007/0283956 A1
(43) Pub. Date: Dec. 13, 2007

(54) Capsule seizing device

(76) Inventor: A-Chuan Hsu, Taishan Township (TW)

Correspondence Address: MACPHERSON KWOK CHEN & HEID LLP, 2033 GATEWAY PLACE, SUITE 400, SAN JOSE, CA, 95110, US

(21) Appl. No.: 11/450,550

(22) Filed: Jun. 12, 2006

(51) Int. Cl. A61M 15/00; B07B 1/00; B65D 83/06; A61M 16/00; B05D 7/14

(52) U.S. Cl. 128/203.15; 209/240

(57) ABSTRACT

A capsule seizing device includes a base section including an air blower to generate an airflow, an airflow channel section in which a guide is arranged to guide the airflow to move in a cyclonic manner, a filter section mounted to an outlet of the airflow channel section, and a hopper mounted to the filter section and having an extension into the airflow channel to guide capsules to be seized into the airflow channel section. Qualified ones of the capsules that are of a weight within a predetermined range are caused to circle around the guide by the cyclonic airflow and discharged through a first discharge opening defined in the airflow channel section. Unqualified capsules that are of a weight below the predetermined range are lifted by the cyclonic airflow into the filter section and discharged through a second discharge opening defined in the filter section. Thus, unqualified capsules are efficiently and effectively separated from the qualified ones.

### BACKGROUND OF THE INVENTION

1. Field of the Invention

The present invention generally relates to a medicine capsule seizing device, and in particular to a capsule seizing device that removes flaw products of medicine capsules in an automatic and efficient manner whereby capsules containing insufficient or even no powder medicine can be effectively removed from the manufacturing lines.

2. The Related Art

Powder medicine or health-care food is often packed in capsules, which allows for quality and quantity control of the medicine or food. However, in the manufacturing process of the capsules, empty capsules or capsules containing insufficient amount are often observed, which capsules must be properly removed for quality control of products and for enhancing subsequent processing.

Manual inspection and removal of the flaw capsules is very inefficient and ineffective. Vibration seizing devices are efficient in removing flaw capsules from the manufacturing line; however, the vibration seizing devices are not effective enough to correctly remove all the flaw products. In addition, damage to the capsules due to wearing caused by friction and contact between the capsules and the seizing device often deteriorates quality of final capsule products.

Thus, it is desired to have an improved and novel capsule seizing device that overcomes the problems discussed above.

### SUMMARY OF THE INVENTION

An objective of the present invention is to provide a capsule seizing device that comprises an air blower that induces an helically moved airflow that lifts capsules of insufficient weight to move helically along a channel whereby, while qualified capsules that are of predetermined weight are allowed to move out of the channel through first discharge opening, unqualified capsules that are of insufficient weights are moved upward by the airflow and discharged through a second opening to be separated from the qualified capsules.

To achieve the above objective, in accordance with the present invention, there is provided a capsule seizing device comprising a base section comprising an air blower, which generates an airflow; an airflow channel section in which an airflow guide is arranged and which defines a first discharge opening, the airflow generated by the air blower being guided to flow through the airflow channel section in a cyclonic manner by the airflow guide; a filter section defining a second discharge opening, the filter section being mounted to an outlet of the airflow channel section to allow the airflow to pass through the filtering screens; a feed hopper fit into the filter section.

The base section comprises a flat-bottom stand and a cover arranged at a predetermined distance from the stand. The air blow is fixed inside the cover. An upright column extends from the stand and through an opening defined in the cover to have an upper free end projecting beyond the cover. A flow product collection tank is fixed on the upper free end of the column for collecting unqualified capsules. A control is provided on the cover for controlling flow rate of the airflow generated by the air blower in a stepless manner.

The airflow channel section comprises a hollow cylinder defining a channel through the airflow passes. The cylinder has a converging conic lower end coupled to an outlet of the air blower to guide the airflow into the channel. A first discharge opening is defined in a cylindrical sidewall of the cylinder to discharge qualified capsules.

The airflow guide comprises a cylindrical body coaxially received and retained in the cylinder of the airflow channel section. The cylindrical body has an upper end forming a convex projection and a lower end forming a converging conic shape. A plurality of spaced and radially extending blades is fixed to the cylindrical body and secured together by an outer rim surrounding the blades. The outer rim forms a cutoff corresponding in position to the first discharge opening of the cylinder of the airflow channel section.

The filter section comprises an annular support having a lower face from which a cylindrical projection extends and an upper face on which an annular inner wall is formed. A central bore is defined through the projection and the inner wall. The cylindrical projection is fit over the outlet of he cylinder of the airflow channel section. A first filtering screen is supported on the support by a plurality of posts having lower ends fixed to the support. The first filtering screen is in a cylindrical configuration to allow for insertion of the hopper and in communication with the bore of the support. The first filtering screen has a lower ring in which an opening is defined. An inclined block is formed on the lower ring adjacent to the opening. The opening of the lower ring of the first filtering screen corresponds in position to a second discharge opening defined in the support. A second filtering screen in a cylindrical shape is supported on the support and surrounds the first filtering screen for removing dusts and powders entraining the airflow. A cap is positioned on the first filtering screen by bolts engaging inner-threaded holes defined in upper ends of the posts with the second filtering screen interposed and retained between the cap and the support. The cap defines a bore for the insertion of the hopper into the filter section. The cap comprises a circumferential skirt that surrounds an outer rim of the support with a gap therebetween for discharging air therethrough.

The hopper comprises a lower extension insertable into the filter section with a lower end entering the channel defined by the cylinder of the airflow channel section and opposing a convex projection formed on an end of the airflow guide to guide the capsules into the airflow channel section.

Capsules are fed into the airflow channel section through the hopper. The airflow generated by the air blower causes the qualified capsules that have a weight within a predetermined range to move around the airflow guide inside the channel and moves through the cutoff of the outer rim to gent into the first discharge opening through which the qualified capsules are discharged.

Unqualified capsules that have a weight below a predetermined range of weight are lifted by the cyclonic airflow into the filter section. The inclined block stops the movement of the unqualified capsules, which causes the unqualified capsules to fall into and move through the second discharge opening. The unqualified capsules are then collected in the flaw product collection tank.

Powders and dusts entraining the airflow can be removed by the second filtering screen. The air may then leave the. seizing device through the gap between the cap and the support of the filter section.

The capsules seizing device makes use of airflow lift to separate the unqualified capsules, which are of less weight due to under-filling and which are thus moved upward by the airflow, from qualified capsules, which are of regular weight and which thus cannot be lifted by the airflow. Effective and efficient separation of flaw products of capsules can be easily and precisely performed.

### BRIEF DESCRIPTION OF THE DRAWINGS

The present invention will be apparent to those skilled in the art by reading the following description of a preferred embodiment thereof, with reference to the attached drawings, in which:

FIG. 1 is a perspective view of a capsule seizing device constructed in accordance with the present invention;

FIG. 2 is an exploded view of the capsule seizing device of the present invention;

FIG. 3 is a side elevational view of the capsule seizing device of the present invention, with a base portion sectioned to show inside details;

FIG. 4 is a side elevational view of the capsule seizing device of the present invention, with a channel portion sectioned to show inside details;

FIG. 5 is a cross-sectional view of a filter section of the capsule seizing device of the present invention; and

FIG. 6 is an exploded view of the filter section.

### DETAILED DESCRIPTION OF THE PREFERRED EMBODIMENT

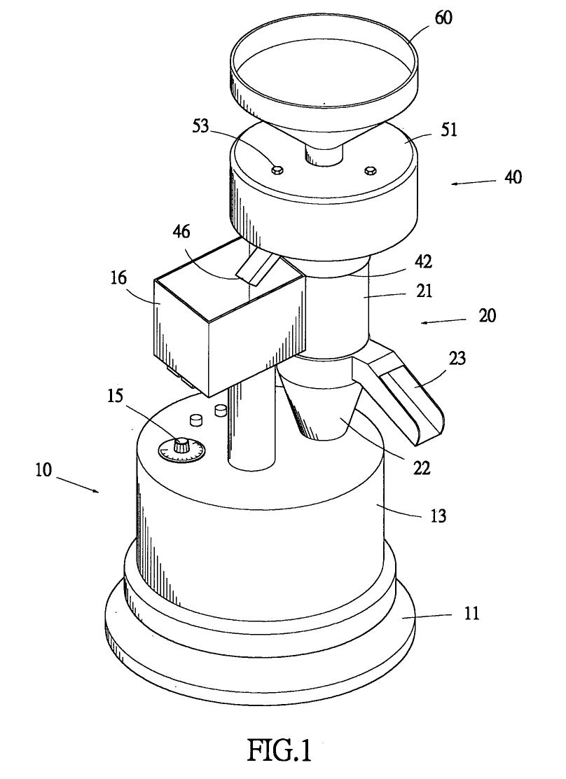

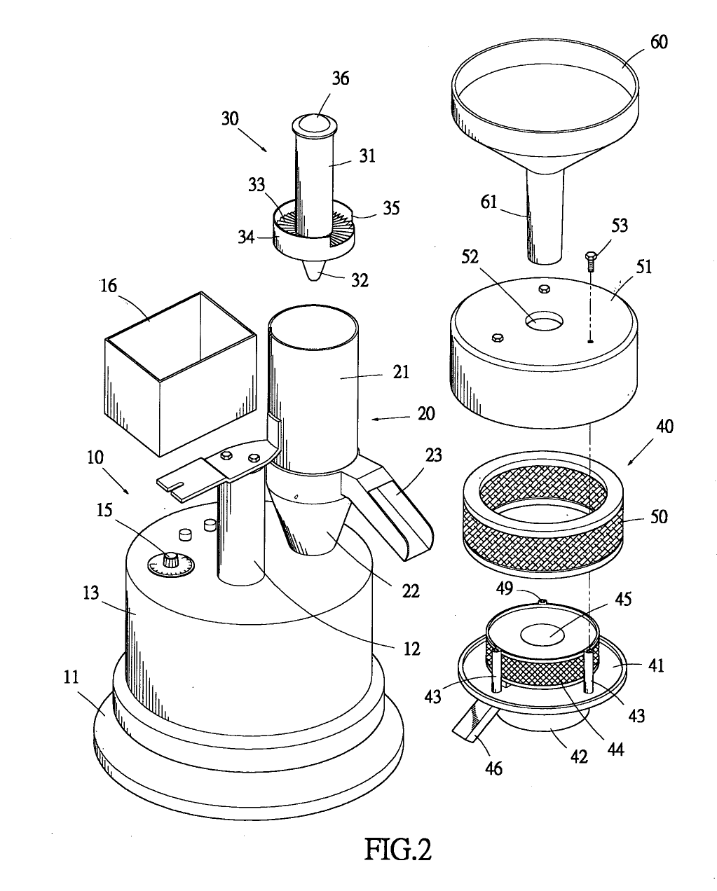

With reference to the drawings and in particular to FIGS. 1 and 2, a capsule seizing device constructed in accordance with the present invention comprises a base section 10 containing an air blower 14, an airflow channel section 20 in which an airflow guide 30 is fixed, a filter section 40 comprising first and second filtering screens 44, 50, and a feed hopper 60.

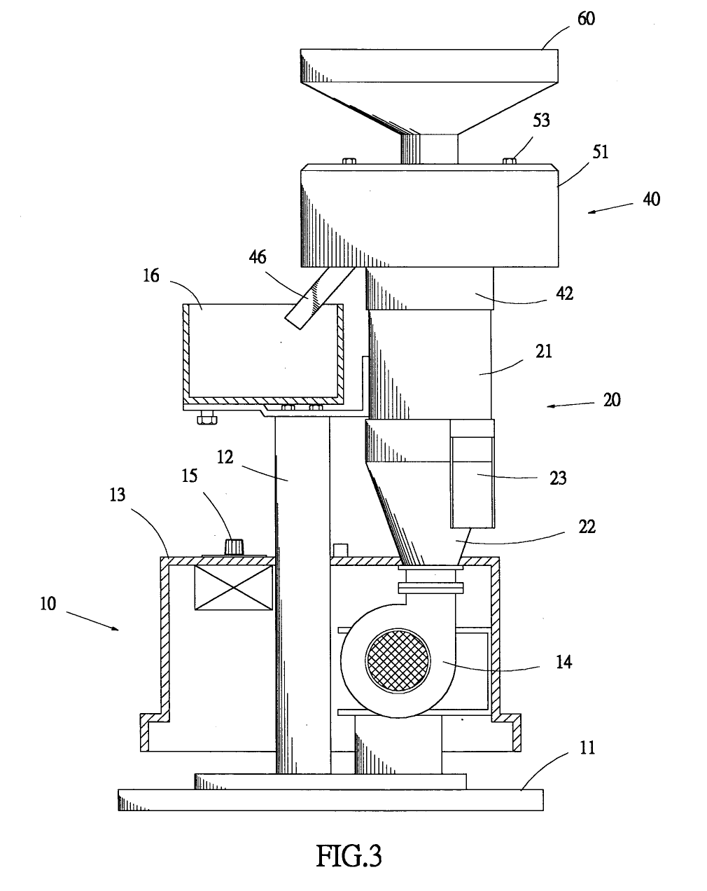

Also referring to FIG. 3, the base section 10 comprises a flat-bottom stand 11 on which a cover 13 is fixed with a gap therebetween. The air blower 14 is fixed inside the cover 13. The stand 11 comprises an upright column 12 extending upward from the top face of the stand 11 and through an opening (not labeled) defined in a top wall of the cover 13 with a top free end of the column beyond and distant from the top wall of the cover 13 to securely support a flaw product collection tank 16. A control 15 is provided on the top wall of the cover 13 to control the flow rate of airflow generated by the air blower 14 in a stepless manner.

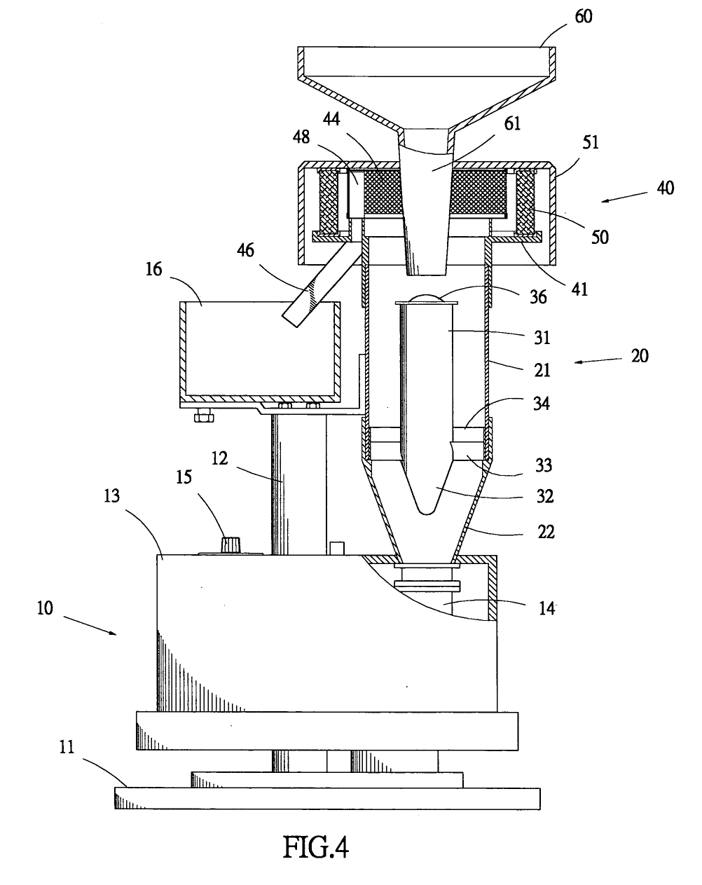

Also referring to FIG. 4, the airflow channel section 20 comprises an external cylinder 21 that is hollow and defines an air channel. The cylinder 21 has a converging conic lower end 22 that extends through an opening defined in the top wall of the cover 13 to connect to an air discharge duct (not labeled) of the air blower 14, whereby the airflow generated by the air blower 14 is guided into the cylinder 21 and along the air channel defined by the cylinder 21. A first capsule discharge opening 23 is defined in the cylinder 21, which will be further described.

The airflow guide 30 comprises a cylindrical body 31 coaxially received and retained in the cylinder 21. The body 31 has a top end forming a convex projection 36 and an opposite lower end 32 that is of converging conic shape, whereby an air passage is formed around the lower conic end 32 and the cylindrical body 31 to allow the airflow from the air blower 14 to flow through the cylinder 21. A blade assembly is mounted to cylindrical body 31 and tightly interposed between the cylindrical body 31 of the airflow guide 30 and the inside surface of the cylinder 21. The blade assembly comprises a plurality of substantially equally spaced and radially extending blades 33 that are arranged at a predetermined inclined angle with respect to a center axis of the cylinder 21. Remote ends of the blades 33 are fixed by a circular outer rim 34 that is in contact with and supported by the inside surface of the cylinder 21. A cutoff 35 is formed in the outer rim 34 at a position corresponding to the first discharge opening 23 of the cylinder 21.

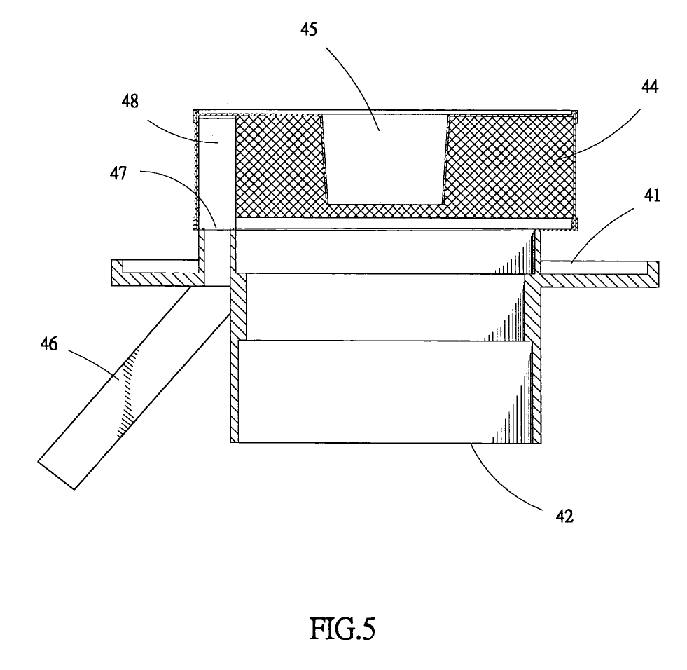

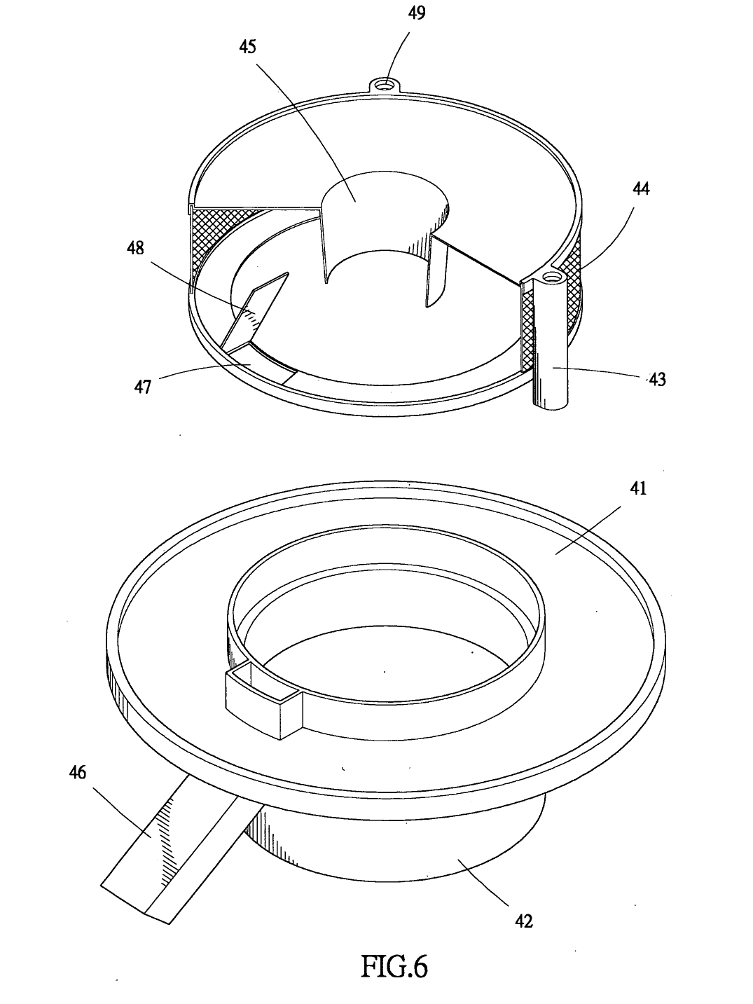

Also referring to FIGS. 5 and 6, the filter section 40 comprises a support 41 in the form of an annular disk having a lower face (not labeled) from which a cylindrical projection 42 extends and an upper face on which an annular inner wall is formed. The inner wall on the upper face and the cylindrical projection 42 on the lower face form a continuous bore extending through the support 41. The lower projection 42 is tightly fit over an outlet defined in an upper end of the cylinder 21 of the airflow channel section 20 to guide the airflow that flows through the channel of the cylinder 21 into the filter section 40.

The first filtering screen 44 is formed as a cylinder supported by posts 43 arranged on an outer circumferential surface of the first filtering screen 44. Lower ends of the posts 43 are fixed to the upper face of the support 41 and upper ends of the posts 43 each define an inner-threaded hole 49. Upper and lower rims of the cylindrical first filtering screen 44 are bounded by upper and lower rings, of which the upper ring defines a central bore 45 that is delimited by a cylindrical inner flange depending from the upper ring. The central bore 45 is sized to receive the insertion of a lower extension 61 of the feed hopper 60 so as to guide capsules to be seized into the cylinder 21 of the airflow channel section 20.

The lower ring of the first filtering screen 44 is positioned on and supported by the annular inner wall of the support 41. An opening 47 is formed in the lower ring and an inclined block 48 is formed on the lower ring adjacent to one side the opening 47 opposite to the direction of airflow generated inside the airflow channel 21. The support 41 forms a second discharge opening 46 corresponding to the opening 47 of the lower ring of the first filtering screen 44 for discharging unqualified capsules that may be of insufficient weight or containing insufficient amount of powder or particle.

The second filtering screen 50 is also made cylindrical, which is positioned on and supported by the upper face of the support 41 and surrounding the first filtering screen 44 for screening off powders and dusts entraining the airflow generated inside the airflow channel section 20.

A cap 51 is mounted to top of the first filtering screen 44 by bolts 53 engaging the inner-threaded hole 49 of the post 43. The second filtering screen 50 is interposed and retained between the cap 51 and the support 41. The cap 51 has a circumferential skirt that surrounds the second filtering screen 50 and an outer circumference of the support 41 with a sufficient gap between the outer circumference of the support 41 and the skirt to allow air to leave the filter section 40.

The extension 61 of the hopper 60 is inserted through a hole 52 defined in a top wall of the cap 50, the central bore 45 of the first filtering screen 44, and the bore of the support 41, with a lower end of the extension 61 entering the upper end of the cylinder 21 and opposing the convex projection 36 of the body 31 of the airflow guide 30. When capsules to be seized are fed into the seizing device through the hopper 60, the capsules are guided by the hopper 60 and the extension 61 into the cylinder 21 and are further guided by the airflow guide 30 to move through the channel defined in the cylinder 20 to eventually reach and pile on the blades 33.

When the air blower 14 is actuated, airflow is caused to flow through the channel defined by the cylinder 21 along a helical or spiral path surrounding the airflow guide 30. The flow rate of the airflow can be set or adjusted by the control 15, in accordance with the predetermined weight of each capsule. Thus, the capsules that are of a weight within a predetermined ranged are moved along the inside surface of the cylinder 21, passing through the cutoff 35 formed in the outer rim 34 and discharged through the first discharge opening 23, while unqualified capsules, which are under-filled or empty at all, are lifted by the cyclonic airflow into the filter section 40. The unqualified capsules are stopped by the block 48 and fall through the opening 47 of the first filtering screen 44 and discharged through the second discharge opening 46 to the flaw product collection tank 16.

Powders and dusts entraining the airflow are filtered off when the air flows through the second filtering screen50 of the filter section 40 to leave the device through the gap between the support 41 and the cap 41.

Making use of cyclonic airflow to separate under-filled and/or empty capsules from well-filled, qualified capsules can minimize damage caused by abrasion between the capsules and the seizing device. In addition, unqualified capsules can be easily lifted to the filter section by the cyclonic airflow for efficient separation of qualified and unqualified capsules. Powders and dusts entraining the airflow can be removed by the second filtering screen 50. Thus, efficient, effective, and clean separation of unqualified or flaw capsules can be done with the present invention.

Although the present invention has been described with reference to the preferred embodiment thereof, it is apparent to those skilled in the art that a variety of modifications and changes may be made without departing from the scope of the present invention which is intended to be defined by the appended claims.

What is claimed is:

1. A capsule seizing device comprising:
a base section comprising an air blower, which generates airflow;
an airflow channel section in which an airflow guide is arranged and which defines a first discharge opening, the airflow generated by the air blower being guided to flow through the airflow channel section in a cyclonic manner by the airflow guide;
a filter section defining a second discharge opening, the filter section being mounted to an outlet of the airflow channel section to allow the airflow to pass through the filtering screens;
a feed hopper fit into the filter section;
wherein capsules are fed into the airflow channel section through the hopper, the airflow lifts first ones of the capsules having a weight below a predetermined range of weight into the filter section to the second discharge opening, while causes second ones of the capsules having a weight within the predetermined range to move to the first discharge opening defined in the airflow channel section.

2. The capsule seizing device as claimed in claim 1, wherein the base section comprises a flat-bottom stand and a cover arranged at a predetermined distance from the stand, the air blow being fixed inside the cover, an upright column extending from the stand and through an opening defined in the cover to have an upper free end projecting beyond the cover, a flow product collection tank fixed on the upper free end of the column for collecting the first capsules that are discharged through the second discharge opening, a control being provided on the cover for controlling flow rate of the airflow generated by the air blower in a stepless manner.

3. The capsule seizing device as claimed in claim 1, wherein the airflow channel section comprises a hollow cylinder defining a channel through the airflow passes, the cylinder having a converging conic lower end coupled to an outlet of the air blower to guide the airflow into the channel, the first discharge opening being defined in a cylindrical sidewall of the cylinder to discharge the second capsules.

4. The capsule seizing device as claimed in claim 1, wherein the airflow guide comprises a cylindrical body coaxially received and retained in the cylinder of the airflow channel section, the body having an upper end forming a convex projection and a lower end forming a converging conic shape, a plurality of spaced and radially extending blades being fixed to the cylindrical body and secured together by an outer rim surrounding the blades, the outer rim forming a cutoff corresponding in position to the first discharge opening of the cylinder of the airflow channel section.

5. The capsule seizing device as claimed in claim 1, wherein the filter section comprising:
an annular support having a lower face from which a cylindrical projection extends and an upper face on which an annular inner wall is formed, a central bore being defined through the. projection and the inner wall, the cylindrical projection being fit over the outlet of he cylinder of the airflow channel section;
a first filtering screen supported on the support by a plurality of posts having lower ends fixed to the support, the first filtering screen being in a cylindrical configuration to allow for insertion of the hopper and in communication with the bore of the support, the first filtering screen having a lower ring in which an opening is defined, an inclined block formed on the lower ring adjacent to the opening, the opening of the lower ring of the first filtering screen corresponding in position to the second discharge opening;
a second filtering screen in a cylindrical shape supported on the support and surrounding the first filtering screen for removing dusts and powders entraining the airflow; and
a cap positioned on the first filtering screen by fasteners engaging inner-threaded holes defined in upper ends of the posts with the second filtering screen interposed and retained between the cap and the support, the cap defining a bore for the insertion of the hopper into the filter section, the cap comprising a circumferential skirt that surrounds an outer rim of the support with a gap therebetween for discharging air therethrough.

6. The capsule seizing device as claimed in claim 1, wherein the hopper comprises a lower extension insertable into the filter section with a lower end entering the channel defined by the cylinder of the airflow channel section and opposing a convex projection formed on an end of the airflow guide to guide the capsules into the airflow channel section.
